(19) United States
(12) Patent Application Publication
(10) Pub. No.: US 2026/0162809 A1
(43) Pub. Date: Jun. 11, 2026

(54) SEGMENTATION METHOD AND DEVICE BASED ON CONDITIONAL DIFFUSION MODEL UTILIZING ANNOTATION MASK

(71) Applicant: SEOUL WOMEN'S UNIVERSITY INDUSTRY-UNIVERSITY COOPERATION FOUNDATION, Seoul (KR)

(72) Inventors: Helen HONG, Seoul (KR); Jin Seo AN, Seoul (KR)

(73) Assignee: SEOUL WOMEN'S UNIVERSITY INDUSTRY-UNIVERSITY COOPERATION FOUNDATION, Seoul (KR)

(21) Appl. No.: 19/411,593

(22) Filed: Dec. 8, 2025

(30) Foreign Application Priority Data
Dec. 9, 2024 (KR) 10-2024-0181496
Nov. 25, 2025 (KR) 10-2025-0180596

(51) Int. Cl. G16H 30/40; G06N 3/0475; G06T 7/00; G06T 7/10; G06V 10/74; G16H 30/20

(52) U.S. Cl. CPC G16H 30/40; G06N 3/0475; G06T 7/0012; G06T 7/10; G06V 10/761; G16H 30/20; G06T 2207/30008

(57) ABSTRACT

The disclosure relates to a medical-image segmentation method and device based on a conditional diffusion model utilizing annotation masks. The device includes a communication module, a memory storing instructions for performing segmentation, and a processor configured to execute the instructions. The processor acquires a medical image of a patient, inputs the image into a diffusion model trained with at least one annotation mask, and generates multiple segmentation results. Based on the multiple results, the processor produces a consensus map representing a degree of agreement among the results. Using the consensus map and/or the acquired medical image, the processor performs correction to refine boundaries and generate a final segmentation result.

### CROSS-REFERENCE TO RELATED APPLICATIONS

A claim for priority under 35 U.S.C. § 119 is made to Korean Patent Application Nos. 10-2024-0181496 filed on Dec. 9, 2024 and 10-2025-0180596 filed on Nov. 25, 2025 in the Korean Intellectual Property Office, the entire contents of which are hereby incorporated by reference.

### BACKGROUND

### 1. Technical Field

The present disclosure relates to a segmentation method and device based on a conditional diffusion model utilizing an annotation mask.

### 2. Description of Related Art

Medical image segmentation is essential for diagnosis, treatment planning, and surgical guidance. However, accurate segmentation is challenging due to complex anatomical structures. Inter-observer variability is common, particularly in cases with thin bones or ambiguous boundaries.

Even when a plurality of specialists annotates the same image, subjective judgments about the structure can lead to differences in result. However, existing approaches to effectively integrating multiple annotations and addressing uncertainty have been limited. Particularly, segmentation of bones with ambiguous boundaries and thin structures is particularly challenging.

Convolutional Neural Network (CNN)-based methods have primarily been used to segment the orbital bone, a thin bone structure. However, these methods have encountered several challenges, including inter-observer variability in generating a single optimal result and limitations in handling ambiguous boundaries. Moreover, the subjectivity of manual labeling often leads to over- or under-segmentation of thin, complex structures, and simple averaging methods risk losing important details.

Therefore, techniques that enable more accurate segmentation of bone structures of various sizes are needed.

### SUMMARY

The present disclosure is to provide a conditional diffusion model-based segmentation device and method utilizing an annotation mask, which segments a medical image utilizing a diffusion model that is effective in learning complex data distribution, thereby enabling precise segmentation, and further improving the segmentation accuracy of bone structures with unclear boundaries.

The problems to be solved by the present disclosure are not limited to the problems mentioned above, and other problems not mentioned will be clearly understood by those skilled in the art from the description below.

In an aspect of the present disclosure, a segmentation device based on a conditional diffusion model utilizing an annotation mask may include a communication module configured to communicating with an external device; a memory configured to store at least one process for performing a segmentation operation based on a conditional diffusion model using an annotation mask; and a processor configured to perform the segmentation operation based on a conditional diffusion model using an annotation mask according to the process, wherein the processor is configured to: collect a medical image acquired by photographing a specific part of a patient, input the medical image into a diffusion model trained based on at least one annotation mask, generate a consensus map indicating a degree of agreement between multiple segmentation results based on the multiple segmentation results being generated from the diffusion model, and perform correction based on at least one of the consensus map or the medical image and generate a final segmentation result.

In another aspect of the present disclosure, a segmentation method based on a conditional diffusion model utilizing an annotation mask performed by a device may include collecting a medical image acquired by photographing a specific part of a patient; inputting the medical image into a diffusion model trained based on at least one annotation mask; generating a consensus map indicating a degree of agreement between multiple segmentation results based on the multiple segmentation results being generated from the diffusion model, and performing correction based on at least one of the consensus map or the medical image and generating a final segmentation result.

In addition, a computer program stored in a computer-readable recording medium for executing a method for implementing the present disclosure may be further provided.

In addition, a computer-readable recording medium recording a computer program for executing a method for implementing the present disclosure may be further provided.

### BRIEF DESCRIPTION OF THE FIGURES

FIG. 1 is a diagram illustrating a network structure of a system for providing a segmentation service based on a conditional diffusion model utilizing an annotation mask according to an embodiment of the present disclosure.

FIG. 2 is a diagram illustrating an example of each annotation mask received from each annotator according to an embodiment of the present disclosure.

FIG. 3 is a diagram schematically illustrating a series of operations for processing a medical image according to an embodiment of the present disclosure.

FIG. 4 is a diagram schematically illustrating a configuration of a service server for providing a segmentation service based on a conditional diffusion model utilizing an annotation mask according to an embodiment of the present disclosure.

FIG. 5 is a diagram schematically illustrating a configuration of a user terminal utilizing a data augmentation service for medical image analysis based on artificial intelligence according to an embodiment of the present disclosure.

FIG. 6 is a diagram illustrating a segmentation method based on a conditional diffusion model utilizing an annotation mask according to an embodiment of the present disclosure.

FIG. 7 is a diagram comparing an example of a medical image segmented according to an embodiment of the present disclosure and a medical image segmented using a method based on the conventional convolutional neural network.

FIG. 8 is a diagram illustrating an effectiveness of medical images generated as final analysis result according to an embodiment of the present disclosure.

### DETAILED DESCRIPTION

The advantages and features of the present disclosure, and methods for achieving them, will become clear with reference to the embodiments described in detail below along with the accompanying drawings. However, the present disclosure is not limited to the embodiments disclosed below and may be implemented in various different forms. The present embodiments are merely provided to ensure that the present disclosure is complete, and provided to fully convey the scope of the present disclosure to those skilled in the art to which the present disclosure pertains. The present disclosure is defined only by the scope of the claims.

The terminology used herein is for the purpose of describing embodiments and is not intended to limit the disclosure. As used herein, singular forms also include plural forms, unless specifically stated otherwise in the context. As used in the specification, “comprises” and/or “comprising” does not exclude the presence or addition of one or more other elements in addition to the mentioned elements. Throughout the specification, the same reference numerals refer to the same elements, and “and/or” includes each and every combination of the elements mentioned. Although “first”, “second”, etc. are used to describe various elements, it is to be understood that these elements are not limited by these terms. These terms are merely used to distinguish one element from another. Accordingly, it should be understood that a first element mentioned below may also be a second element within the technical scope of the present invention.

Unless otherwise defined, all terms (including technical and scientific terms) used in this specification may be used with meanings commonly understood by those skilled in the art to which this disclosure pertains. Additionally, terms defined in commonly used dictionaries are not to be interpreted ideally or excessively unless clearly specifically defined.

The same reference numerals refer to the elements throughout the present disclosure. This disclosure describes not all elements of the embodiments, and general descriptions or redundant descriptions in the embodiments in the technical field to which this disclosure belongs are omitted. The term “unit” or “module” used in the specification means a software, hardware component such as an FPGA or an ASIC, and the “unit” or “module” performs certain roles. However, the “unit” or “module” is not limited to software or hardware. The “unit” or “module” may be configured to be in an addressable storage medium and may be configured to play one or more processors. Thus, as an example, the “unit” or “module” includes components such as software components, object-oriented software components, class components, and task components, and processes, functions, properties, procedures, subroutines, segments of program code, drivers, firmware, microcode, circuits, data, databases, data structures, tables, arrays, and variables. The functions provided within the components and “parts” or “modules” may be combined into a smaller number of components and “parts” or “modules” or further separated into additional components and “parts” or “modules”.

Throughout the specification, when a part is said to be “connected” to another part, this includes not only the case where it is directly connected, but also the case where it is indirectly connected, and the indirect connection includes the connection via a wireless communication network.

Also, when a part is said to “include” a component, this does not mean that other components are excluded, unless otherwise specifically stated, but that other components may be included.

Throughout the specification, when a component is said to be “on” another component, this includes not only the case where the component is in contact with the other component, but also the case where another component exists between the two components.

The terms first, second, and the like are used to distinguish one component from another component, and the components are not limited by the aforementioned terms.

A singular expression includes a plural expression unless there is an obvious exception in the description.

The identifiers in each step are used for convenience of description and do not describe the order of each step, and each step may be performed in a different order than the stated order unless the description clearly describes a specific order.

The terms used in the following description are defined as follows.

Although described herein as a “service server,” this is a device for providing a pediatric brain perivascular space data augmentation service based on medical images, and may include various devices capable of performing computational processing. In addition, this service server may be connected/interlocked with a separate server, computer, and/or portable terminal to change settings or collect or analyze information and provide it, and its type and form are not limited.

The computer may include, for example, a notebook, a desktop, a laptop, a tablet PC, a slate PC, and the like equipped with a web browser.

Here, the server is a server that communicates with an external device to process information, and may include an application server, a computing server, a database server, a file server, a game server, a mail server, a proxy server, and a web server.

The portable terminal may include, for example, a wireless communication device that ensures portability and mobility, such as a PCS (Personal Communication System), a GSM (Global System for Mobile communications), a PDC (Personal Digital Cellular), a PHS (Personal Handyphone System), a PDA (Personal Digital Assistant), an IMT (International Mobile Telecommunication)-2000, a CDMA (Code Division Multiple Access)-2000, a W-CDMA (W-Code Division Multiple Access), a WiBro (Wireless Broadband Internet) terminal, a smart phone, and all kinds of handheld-based wireless communication devices, as well as wearable devices such as watches, rings, bracelets, anklets, necklaces, glasses, contact lenses, or head-mounted devices (HMDs).

In this specification, the artificial intelligence-based pre-learning model or service platform according to the present disclosure may be generated and provided by a computing device based on big data and artificial intelligence (AI) technology, and can be implemented using or referencing ICT (Information and Communication Technology) technologies such as virtual reality (VR), augmented reality (AR), and mixed reality (MR), extended Reality (XR), and blockchain technology for the security of medical information or personal information included therein. However, in this specification, a detailed description of the ICT technology related to the present disclosure is omitted by referring to known technologies.

Hereinafter, the operating principle and embodiments of the present disclosure will be described with reference to the attached drawings.

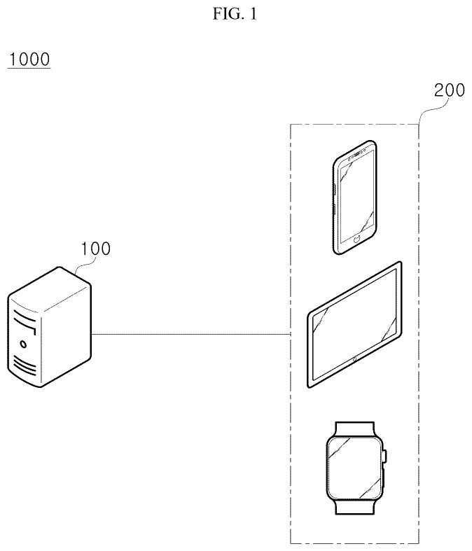

FIG. 1 is a diagram illustrating a network structure of a system for providing a segmentation service based on a conditional diffusion model utilizing an annotation mask according to an embodiment of the present disclosure.

Referring to FIG. 1, a system 1000 for providing a segmentation service based on a conditional diffusion model utilizing an annotation mask according to an embodiment of the present disclosure (hereinafter referred to as a “service providing system”) may be configured to include at least one of a service server 100 or a user terminal 200.

The service server 100 is a device for providing a segmentation service based on a conditional diffusion model utilizing an annotation mask according to an embodiment of the present disclosure (hereinafter referred to as a “segmentation service”), that is, a segmentation service providing device. For this purpose, the service server 100 may provide a separate web page and/or service platform (application), and each user terminal 200 may provide or receive various information/data for the segmentation service based on the web page or service platform.

In addition, the service server 100 transmits and receives various data and/or information with the user terminal 200 or a separate external device (not shown) based on at least one communication method, and provides the user with the segmentation service.

To provide the segmentation service, the service server 100 is connected to the user terminal 200 and may collect or receive all information/data related to the segmentation service, such as various notifications, requests, and information. Meanwhile, the service providing system 1000 may be linked (connected) to an electronic medical record system to collect and utilize various information for each patient. The electronic medical record system is a system configured to enable each medical professional or each medical institution to record and manage various information for each patient. When information for a specific patient is entered from a specific medical professional or a specific medical institution, all of this may be processed and managed as a database. Here, various information for each patient may include basic information such as personal information and physical information, as well as medical data (medical information) including at least one of medical history, treatment result, test result, or medical images. However, this is merely an example, and other additional information for the patient may be included or at least some of it may be excluded, and the stored data/information is not limited to the data/information listed above.

Accordingly, the service server 100 collects at least one piece of medical data through the user terminal 200 and/or the electronic medical record system. In the present disclosure, segmentation of various bone structures is performed based on the patient's medical image. For this purpose, a medical image of the patient may be collected as at least one piece of medical data. The medical image may be, for example, a CT image or a magnetic resonance image.

Meanwhile, the service server 100 may also collect at least a portion of the at least one piece of medical data through a crawler or an Application Programming Interface (API). The medical data to be collected may be identified through data tagging. The crawler is a type of data collector that automatically searches and indexes various information on the web. The crawler is also referred to as software, a spider, a bot, and an intelligent agent. The crawler continuously searches for new web pages according to pre-programmed computer program methods and repeatedly retrieves new information based on the search result. The API refers to a collection of screen layouts and various functions necessary for application developers to easily develop programs that run on an operating system. Utilizing the API allows for the real-time collection of medical data about patients, which is generated or updated frequently through at least one external device/server.

Specifically, when the service server 100 receives a service provision request from the user terminal 200 requesting a segmentation service for a medical image of a patient, the service server 100 may segment various bone structures based on the medical image taken of a specific area of the patient. Accordingly, the service server 100 may generate and provide, in response to the service provision request, not only the segmentation result but also analysis information based on the segmentation result to the user terminal 200.

In this case, the analysis information may be generated based on a pre-stored template, such that the analysis result based on the segmentation result are provided in at least one of a text, a number, a special character, an emoticon, an image, or an audio, and may also be provided using a graph or a color. In other words, the analysis information may be provided in various ways and formats, and the method and/or format of provision is not limited.

To provide the segmentation service, the service server 100 may construct and equip at least one artificial intelligence-based pre-training model. Furthermore, the service server 100 may continuously manage, that is, retrain and/or update, the constructed at least one learning model to improve its performance.

Meanwhile, the user terminal 200 is a terminal possessed by a user who has been pre-registered with the service server 100 to use the segmentation service provided by the service server 100, and may include at least one terminal respectively. For example, the user may be a medical professional (doctor, nurse, nursing assistant, administrator, etc.) or a medical institution that has the authority to access or share the medical data of the corresponding pediatric patient. That is, each of the at least one user terminal 200 is the same in that it is a terminal possessed by the user, but may be distinguished according to its function or authority. Meanwhile, to use the segmentation service provided by the service server 100, each user may access a web page provided by the service server 100 through the user terminal 200 possessed by the user, or may install and provide a service platform (application).

To utilize the segmentation service provided by the service server 100, each user may access a web page provided by the service server 100 through their own user terminal 200, or install a platform (application). However, for the convenience of description, the following description will be limited to cases where the segmentation service is provided through the service platform.

Based on this platform, each user transmits a service provision request to the service server 100 through the user terminal 200, and in response, the service server 100 may provide not only the quantification result for the pediatric patient, but also developmental prediction information based on the quantification result. This may be determined by allowing the user to set the information they wish to receive when requesting service provision.

Accordingly, the user terminal 200 may display the quantification result and/or development prediction information on a display module equipped in the user terminal 200 or connected separately so that the user may easily visually confirm it.

Meanwhile, the each user terminal 200 may be a computer, UMPC (Ultra Mobile PC), workstation, netbook, PDA (Personal Digital Assistants), portable computer, web tablet, wireless phone, mobile phone, smart phone, pad, smart watch, wearable terminal, e-book, PMP (portable multimedia player), portable game console, navigation device, black box or digital camera, other mobile communication terminal, etc., on which each user may install and execute multiple application programs (i.e., applications) desired. That is, the each user terminal 200 may be provided in various forms, and its form, number, and type are not limited.

As described above, the service providing system 1000 according to the present disclosure may be implemented through data/information transmission and reception between the service server 100 and the user terminal 200 based on network.

As described above, although not illustrated in FIG. 1, the service providing system 1000 may further include, in addition to the user terminal 200, at least one patient terminal (not illustrated) and/or at least one guardian terminal (not illustrated) provided by a pediatric patient.

In this case, each patient terminal and/or each guardian terminal may also receive quantification result and/or developmental prediction information from the service server 100 based on a platform or separate web page, and display the same on a display module, thereby allowing each patient and/or each guardian to easily visually confirm the result. In this case, the quantification result and/or developmental prediction information provided to each patient terminal and/or each guardian terminal may also be provided in various ways and formats, and the method and/or format of provision is not limited. In this case, the quantification result and/or developmental prediction information provided to each patient and/or each guardian may be edited or processed via the user terminal 200. For example, the quantification result and/or developmental prediction information may further include medical opinions recorded during editing or processing by a medical professional.

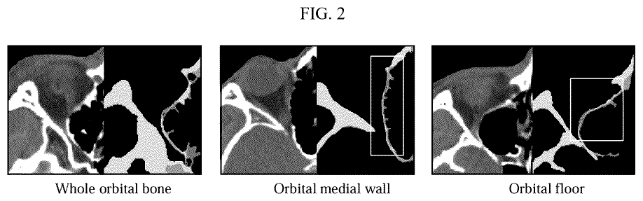

FIG. 2 is a diagram illustrating an example of each annotation mask received from each annotator according to an embodiment of the present disclosure.

First, the medical image segmentation is essential for diagnosis, treatment planning, and surgical guidance, but accurate segmentation remains a challenging task due to complex anatomical structures. Particularly, inter-observer variability in manually annotated masks presents a significant challenge in the medical image segmentation, resulting from differences in expertise, experience, and subjective determination among multiple annotators.

Referring to FIG. 2, it may be seen that each annotator manually generated annotation masks, and inter-observer variability exists due to differences in orbital bone shape between patients, even when following the same annotation guidelines.

Here, FIG. 2 shows annotation masks generated by each annotator (expert) for the entire orbital bone, the inner orbit, and the orbital floor, respectively. Due to the nature of the bone structure, which is composed of very thin, low-strength bone, its variability may be determined to be even greater.

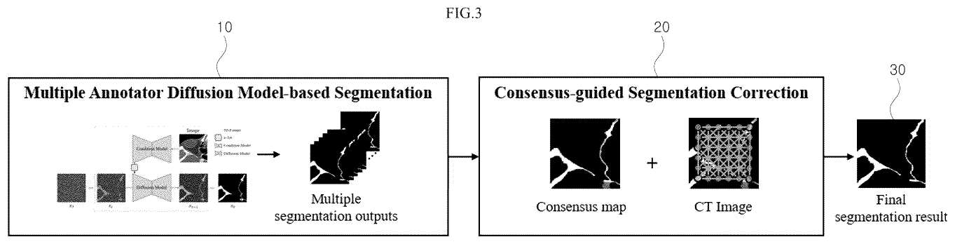

FIG. 3 is a diagram schematically illustrating a series of operations for processing a medical image according to an embodiment of the present disclosure.

Referring to FIG. 3, a medical image may be processed in two steps (Steps 1 and 2) according to an embodiment of the present disclosure.

First, in Step 1, during a multiple annotator diffusion model-based segmentation 10, a medical image is input and multiple segmentation results are output. In this case, the inter-observer variability of the manual annotation mask in ambiguous areas need to be considered.

To achieve this, a diffusion model is trained using various annotation masks obtained from three annotators with different expertise. The probabilistic nature of the diffusion model is then leveraged to output multiple segmentation results for each medical image, such as a CT image.

The backbone of the diffusion model may be MedSegDiff-v2, a transformer-based conditional diffusion model that uses CT image features as conditional information.

Furthermore, it is effective in modeling variability between annotation masks and capturing the uncertainty inherent in complex and ambiguous anatomical structures.

Subsequently, in Step 2, a consensus-guided segmentation correction 20, correction is performed using at least one of the consensus map or the spatial feature of the medical image to address differences between the multiple segmentation results generated by the diffusion model.

Here, the consensus map is generated by aggregating multiple segmentation outputs representing the level of agreement (range 0.0 to 1.0).

Meanwhile, the final segmentation result is modified by minimizing an energy function. First, the probability value derived from the consensus map is used to reflect the segmentation agreement of each pixel, and at least one pairwise feature of the consensus map and the medical image, including spatial proximity, intensity similarity, and gradient directionality, is considered.

Thus, the final segmentation result 30 may be output.

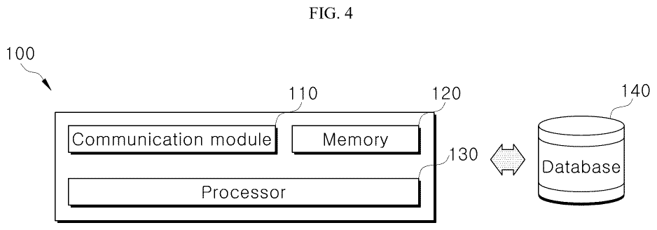

FIG. 4 is a diagram schematically illustrating a configuration of a service server for providing a segmentation service based on a conditional diffusion model utilizing an annotation mask according to an embodiment of the present disclosure.

Referring to FIG. 4, the service server 100 according to an embodiment of the present disclosure may include at least one of a communication module 110, a memory 120, and a processor 130.

The communication module 110 may communicate with at least one of various terminals (devices), external storage (e.g., a database 140), an external server, or a cloud server.

Meanwhile, the external server or the cloud server may be configured to perform at least a portion of the role of the processor 130. That is, data processing or data operations may be performed on the external server or the cloud server, and the present disclosure does not place any particular limitations on such methods.

Meanwhile, the communication module 110 may support various communication schemes depending on the communication standards of the communication target (e.g., electronic device, external server, device, etc.).

For example, the communication module 110 may be configured to communicate with a communication target using at least one of WLAN (Wireless LAN), Wi-Fi (Wireless-Fidelity), Wi-Fi (Wireless Fidelity) Direct, DLNA (Digital Living Network Alliance), WiBro (Wireless Broadband), WiMAX (World Interoperability for Microwave Access), HSDPA (High Speed Downlink Packet Access), HSUPA (High Speed Uplink Packet Access), LTE (Long Term Evolution), LTE-A (Long Term Evolution-Advanced), 5G (5th Generation Mobile Telecommunication), Bluetooth™, RFID (Radio Frequency Identification), IrDA (Infrared Data Association), UWB (Ultra-Wideband), ZigBee, NFC (Near Field Communication), Wi-Fi Direct, or Wireless USB (Wireless Universal Serial Bus) technologies.

Meanwhile, the memory 120 may be configured to store various information related to the present disclosure. In the present disclosure, the memory 120 may be provided in the device itself according to the present disclosure. Alternatively, at least a portion of the memory 120 may refer to at least one of the database (DB, 140 and the cloud storage (or cloud server). That is, the memory 120 may be sufficient as long as it is a space where information required for the device and method according to the present disclosure is stored, and it may be understood that there are no restrictions on the physical space. Accordingly, hereinafter, the memory 120, the database 140, the external storage, and the cloud storage (or cloud server) will not be separately distinguished, and will all be referred to as the memory 120.

The memory 120 may store a plurality of application programs (or applications) running on the service server 100, data for the operation of the service server 100, and commands. At least some of these application programs may be downloaded from an external server via wireless communication. Meanwhile, the application programs may be stored in at least one memory provided in the memory 120, installed on the service server 100, and driven to perform operations (or functions) by at least one processor stored in the memory 120 via the processor 130.

Meanwhile, at least one memory may include at least one type of storage medium including a flash memory type, a hard disk type, a multimedia card micro type, a card type memory (e.g., an SD or XD memory, etc.), random access memory (RAM), static random access memory (SRAM), read-only memory (ROM), electrically erasable programmable read/write (EEPROM), Programmable Read-Only Memory (PROM), a Programmable Read-Only Memory (PROM), a magnetic memory, a magnetic disk, and an optical disk. In addition, the memory may store information temporarily, permanently, or semi-permanently, and may be provided as a built-in or removable type.

Next, the processor 130 may be configured to control the overall operation of the device related to the present disclosure. The processor 130 may process signals, data, information, and the like input or output through the components described above, or provide or process appropriate information or functions to a user (including a manager, a worker, an inspector, etc.).

The processor 130 may include at least one Central Processing Unit (CPU) and perform functions according to the present disclosure.

Specifically, the processor 130 may be configured to collect a medical image acquired by photographing a specific part of a patient, input the medical image into a diffusion model trained based on at least one annotation mask, and then, based on the multiple segmentation results being generated from the diffusion model, generate a consensus map indicating a degree of agreement between multiple segmentation results. Thereafter, the processor 130 may be configured to perform correction based on at least one of the consensus map or the medical image and generate a final segmentation result.

Here, at least one annotation mask may be collected by a single annotator or by multiple annotators. That is, at least one annotation mask may be received by a single annotator, or at least one annotation mask may be received from each of a plurality of annotators and utilized for training the diffusion model, and the receiving method thereof is not limited.

In this case, the consensus map represents the degree of agreement of each pixel by integrating the multiple segmentation result generated by the diffusion model, and may be converted into a probability map and utilized.

Furthermore, the diffusion model is a transformer-based conditional diffusion model that uses an image feature of the medical image as conditional information, and may extract diffusion noise and semantic feature through a conditional U-Net framework.

Meanwhile, the processor 130 may be configured to generate the final segmentation result by performing correction by minimizing an energy function.

For example, when performing correction based on the consensus map and the feature of the medical image to be segmented, the processor 130 may be configured to reflect the segmentation match based on the probability of each pixel that may be extracted from the consensus map. Furthermore, the processor may be configured to apply at least one pairwise feature of the consensus map or the medical image, including at least one of spatial proximity, intensity similarity, or gradient directionality.

In this way, the processor 130 may construct and be provided with at least one AI-based pre-learning model to provide the segmentation service, and may provide the model to the user terminal 200 and/or other external terminals/devices (not shown) that wish to use the segmentation service.

Specifically, the processor 130 may generate at least one AI-based pre-learning model based on medical images (CT images, magnetic resonance images, etc.) collected from at least one server, device, terminal, and the like for the segmentation service. At this time, big data and AI technologies may be used to generate each learning model. For example, each learning model may be generated by constructing a learning data set based on medical images collected from at least one server, device, terminal, and the like, and performing learning on the original model through the data set.

To build a pre-training model in this way, the processor 130 may collect medical images, that is, magnetic resonance images, for each of a plurality of pediatric patients over a preset period of time, preprocess each magnetic resonance image, and label the images based on their resolution, thereby generating a training data set. For example, the processor 130 may perform pre-processing based on at least one of Min-Max scaling, standard scaling, MaxAbs, Normalizer, Quantile Transformer, and Power Transformer.

Furthermore, when the processor 130 receives feedback information on segmentation result and/or analysis information from the user terminal 200, the processor 130 may retrain and update the built pre-training model based on this feedback information, thereby improving the performance of the pre-training model to provide segmentation result and/or analysis information more suitable for the patient.

In addition, the specific operation of the processor 130 will be described below based on each drawing.

Furthermore, at least one component may be added or deleted depending on the performance of the components illustrated in FIG. 4. Furthermore, it will be readily apparent to those skilled in the art that the mutual positions of the components may be changed depending on the performance or structure of the device.

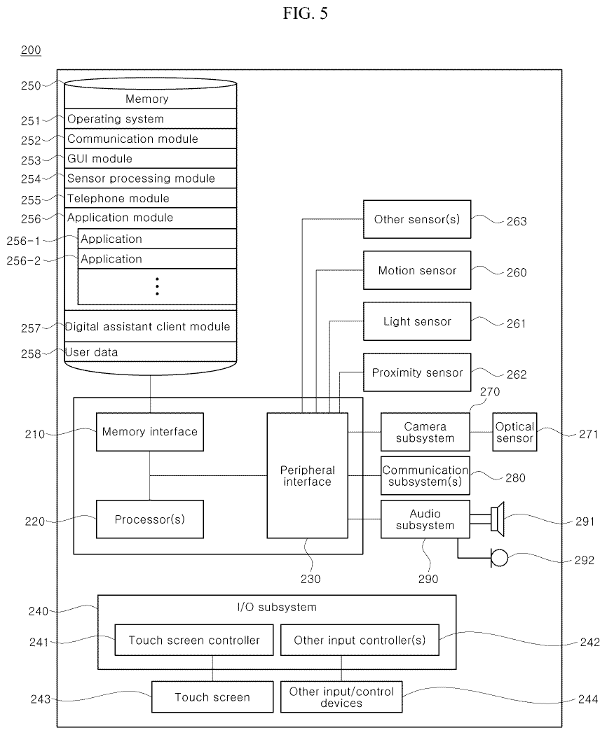

FIG. 5 is a diagram schematically illustrating a configuration of a user terminal utilizing a data augmentation service for medical image analysis based on artificial intelligence according to an embodiment of the present disclosure.

Referring to FIG. 5, the user terminal 200 may include a memory interface 210, one or more processors 220, and a peripheral interface 230. Various components within the user terminal 200 may be connected via one or more communication buses or signal lines.

The memory interface 210 may be connected to the memory 250 to transmit various data to the processor 220. Here, the memory 250 may include at least one type of storage medium among flash memory, hard disk, multimedia card micro, card-type memory (e.g., SD or XD memory), RAM, SRAM, ROM, EEPROM, PROM, network storage, cloud, and blockchain database.

In various embodiments, the memory 250 may store a web/app application or program for utilizing a data augmentation service. In addition, the memory 250 may store various types of information regarding at least one medical professional, patient, guardian, and the like, and may store various types of information acquired through the application or program.

In various embodiments, the memory 250 may store at least one of an operating system 251, a communication module 252, a graphical user interface module (GUI) 253, a sensor processing module 254, a telephone module 255, and an application module 256. Specifically, the operating system 251 may include instructions for processing basic system services and instructions for performing hardware operations. The communication module 252 may communicate with at least one of one or more other devices, computers, and servers. The graphical user interface module (GUI) 253 may process a graphical user interface. The sensor processing module 254 may process sensor-related functions (e.g., processing voice input received through one or more microphones 292. The telephone module 255 may process telephone-related functions. The application module 256 may perform various functions of a user application, such as electronic messaging, web browsing, media processing, navigation, imaging, and other processing functions. In addition, the user terminal 200 may store one or more software applications 256-1 and 256-2 (e.g., service applications) associated with a type of service in the memory 250.

In various embodiments, the memory 250 may store a digital assistant client module 257 (hereinafter, DA client module), and accordingly, may store commands for performing client-side functions of the digital assistant and various user data 258 (e.g., patient-tailored vocabulary data, preference data, other data such as the user's electronic address book, etc.).

Meanwhile, the DA client module 257 may obtain a user's voice input, text input, touch input, and/or gesture input through various user interfaces (e.g., I/O subsystem 240) provided in the user terminal 200.

In addition, the DA client module 257 may output data in audiovisual and tactile forms. For example, the DA client module 257 may output data consisting of a combination of at least two of voice, sound, notification, text message, menu, graphic, video, animation, and vibration. In addition, the DA client module 257 may communicate with a digital assistant server (not shown) using a communication subsystem 280.

In various embodiments, the DA client module 257 may collect additional information for the surrounding environment of the user terminal 200 from various sensors, subsystems, and peripheral devices to construct a context associated with the user input. For example, the DA client module 257 may provide context information along with the user input to the digital assistant server to infer the user's intent. Here, contextual information that may accompany user input may include sensor information, such as lighting, ambient noise, ambient temperature, images of the surrounding environment, video, and the like. For another example, the context information may include the physical state of the user terminal 200 (e.g., device orientation, device location, device temperature, power level, speed, acceleration, motion pattern, cellular signal strength, etc.). For another example, the context information may include information related to the software state of the user terminal 200 (e.g., processes running on the user terminal 200, installed programs, past and present network activity, background services, error logs, resource usage, etc.).

In various embodiments, the memory 250 may include added or deleted instructions. Furthermore, the user terminal 200 may also include additional components in addition to the components illustrated in FIG. 4, or may exclude some components.

The processor 220 may control the overall operation of the user terminal 200 and execute various commands to utilize the data augmentation service provided by the service server 100 by running applications or programs stored in the memory 250.

The processor 220 may correspond to a computing device such as a Central Processing Unit (CPU) or an Application Processor (AP). Furthermore, the processor 220 may be implemented in the form of an integrated chip (IC), such as a System on Chip (SoC) that integrates various computing devices that perform machine learning, such as an NPU (Neural Processing Unit).

In various embodiments, the processor 220 may provide various information and/or data to the user based on the platform through a user interface screen.

The peripheral interface 230 may be connected to various sensors, subsystems, and peripheral devices, and may provide data so that the user terminal 200 may perform various functions. Here, the user terminal 200 performing a certain function may be understood as being performed by the processor 220.

The peripheral interface 230 may receive data from a motion sensor 260, a light sensor (optical sensor) 261, and a proximity sensor 262, through which the user terminal 200 may perform orientation, light, and proximity detection functions, etc. For another example, the peripheral interface 230 may receive data from other sensors 263 (positioning system-GPS receiver, temperature sensor, biometric sensor), through which the user terminal 200 may perform functions related to the other sensors 263.

In various embodiments, the user terminal 200 may include a camera subsystem 270 connected to the peripheral interface 230 and an optical sensor 271 connected thereto, through which the user terminal 200 may perform various photographing functions, such as taking pictures and recording video clips. At this time, the camera subsystem 270 may be provided as a single photographing module including at least one photographing device. Here, the at least one photographing device may be configured to include at least one of a 2D camera, a 3D camera, a ToF (Time of Flight) camera, a light field camera, a stereo camera, an event camera, an infrared camera, a lidar sensor, or an array camera.

In various embodiments, the user terminal 200 may include a communication subsystem 280 connected to the peripheral interface 230. The communication subsystem 280 may be configured with one or more wired/wireless networks and may include various communication ports, radio frequency transceivers, and optical transceivers.

In various embodiments, the user terminal 200 includes an audio subsystem 290 connected to the peripheral interface 230, and the audio subsystem 290 includes one or more speakers 291 and one or more microphones 292, thereby enabling the user terminal 200 to perform voice-activated functions, such as voice recognition, voice duplication, digital recording, and telephone functions.

In various embodiments, the user terminal 200 may include an I/O subsystem 240 connected to the peripheral interface 230. For example, the I/O subsystem 240 may control a touch screen 243 included in the user terminal 200 via a touch screen controller 241.

For example, the touch screen controller 241 may detect a user's contact and movement or cessation of contact and movement using any one of a plurality of touch sensing technologies such as capacitive, resistive, infrared, surface acoustic wave technology, proximity sensor array, and the like. In another example, the I/O subsystem 240 may control other inputs. Other input/control devices 244 included in the user terminal 200 may be controlled through the controller(s) 242. For example, the other input controller(s) 242 may control one or more buttons, rocker switches, thumb wheels, infrared ports, USB ports, and pointer devices such as a stylus.

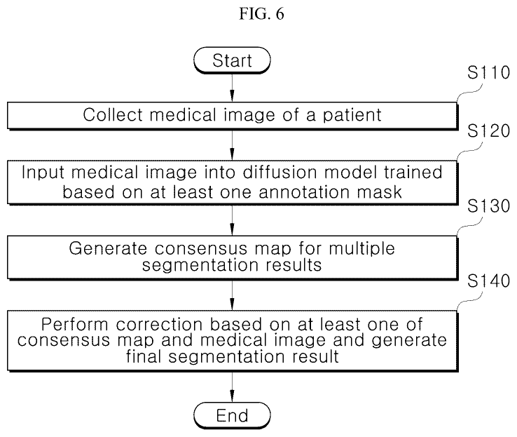

FIG. 6 is a diagram illustrating a segmentation method based on a conditional diffusion model utilizing an annotation mask according to an embodiment of the present disclosure.

Referring to FIG. 6, the service server 100 collects a medical image acquired by photographing a specific part of a patient (step S110) and inputs the medical image into a diffusion model trained based on at least one annotation mask (step S120). In this case, the medical image is acquired by photographing a specific part of the patient using at least one imaging technique, such as CT or MR. The imaging technique used to acquire the medical image may be determined based on the specific part. For example, when segmenting the orbital bone, a facial CT image may be acquired and utilized.

Here, the diffusion model may be configured to probabilistically convert the class value of each pixel into a binary state of 0 or 1 using Bernoulli noise in a discrete probability space, rather than a continuous normal distribution.

Accordingly, in the learning step, each annotation mask collected from multiple annotators is input, and the pixel-level probability value of each mask is sampled from a Bernoulli distribution. In the reverse diffusion phase, the anatomical intensity distribution of the CT image is provided as condition information, thereby simultaneously removing noise and restoring the structural shape.

The Bernoulli sampling-based diffusion model directly reflects the binary mask characteristic of the mask, thereby improving binary segmentation accuracy even in complex medical images and suppressing the boundary blending (blur) phenomenon that occurs in continuous diffusion models.

Next, when the service server 100 generates multiple segmentation results from the diffusion model in step S120, the service server 100 generates a consensus map indicating the degree of agreement for the generated multiple segmentation results (step S130).

Next, the service server 100 performs correction based on the consensus map generated in step S130 and generate a final segmentation result (step S140).

In this case, the processor 130 of the service server 100 may be configured to perform correction by minimizing the energy function defined in [Equation 1] below, based on the probability value of each pixel derived from at least one of the consensus map or the medical image.

$$E ( x ) = {\sum }_{i} {ψ}_{i} ( {x}_{i} ) + {\sum }_{i , j} {ψ}_{ij} ( {x}_{i} , {x}_{j} ) \qquad [ Equation 1 ]$$

Here, ψᵢ(xᵢ)=−log P(xᵢ) denotes a unary potential for each pixel, which may be defined as a weighted value obtained by logarithmically inversely transforming the pixel matching probability on the consensus map and weighting its reliability.

In addition,

$${ψ}_{ij} ( {x}_{i} , {x}_{j} ) = μ ( {x}_{i} , {x}_{j} ) [ {w}_{1} exp ( - \frac{{\vert {p}_{i} , {p}_{j} \vert}^{2}}{2 {θ}_{α}^{2}} - \frac{{\vert {c}_{i} , {c}_{j} \vert}^{2}}{2 {θ}_{β}^{2}} - \frac{{\vert {d}_{i} , {d}_{j} \vert}^{2}}{2 {θ}_{γ}^{2}} ) + {w}_{2} exp ( - \frac{{\vert {p}_{i} , {p}_{j} \vert}^{2}}{2 {θ}_{δ}^{2}} ) ]$$

represents a pairwise potential, pᵢ,pⱼ represents the spatial coordinate of a pixel, cᵢ,cⱼ represents the consensus level, and dᵢ,dⱼ represents the gradient direction. μ(xᵢ,xⱼ) is a class compatibility function according to the Potts model, which may be set to 1 in the case that two pixels are assigned to different classes and 0 in the case that two pixels are the same.

In addition, the control parameters θ_{α},θ_{β},θ_{γ}, θ_{δ} are parameters that determine spatial proximity, consensus level, directional similarity, and correction range, respectively, and may be set to a range of 2 to 100 depending on the image size and pixel resolution, and may be configured as, for example, θ_{α}=80, θ_{β}=60, θ_{γ}=2, θ_{δ}=3.

In addition, the weights may be set to 15 and 1, respectively, and may be configured to adjust the balance of multiple similarity terms.

By minimizing the energy function defined as described above, the service server 100 may prevent discontinuity in thin structures by integrating spatially adjacent pixels with similar consensus levels and directionality into the same class region.

Furthermore, the service server 100 may perform correction based on the consensus map generated in step S130 and the spatial feature of the medical image to be segmented and generate the final segmentation result.

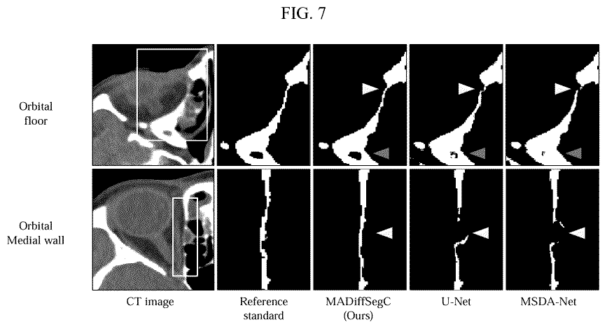

FIG. 7 is a diagram comparing an example of a medical image segmented according to an embodiment of the present disclosure and a medical image segmented using a method based on the conventional convolutional neural network.

As illustrated in FIG. 7, the quantitative evaluation result for orbital bone segmentation obtained using the method (Ours) according to the present disclosure shows significantly improved accuracy compared to the method based on the conventional convolutional neural network, effectively reducing false positives.

Furthermore, the consensus-guided segmentation correction is found to be more effective than simple averaging in utilizing multiple segmentation outputs in a diffusion model. That is, the segmentation performance improved as the number of generated segmentation outputs increased.

Meanwhile, the qualitative evaluation result for orbital bone segmentation obtained using the method according to the present disclosure shows the result more similar to the reference standard compared to the method based on the conventional convolutional neural network.

While the method based on the conventional convolutional neural network tends to over- and under-analyze segmentations, the method according to the present disclosure reduces such false positives and negatives.

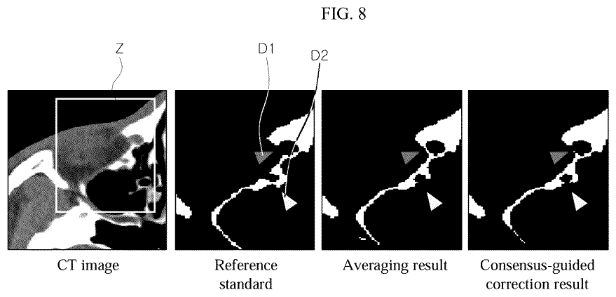

FIG. 8 is a diagram illustrating an effectiveness of medical images generated as final analysis result according to an embodiment of the present disclosure.

The conventional methods have limitations in the ambiguous region (Z), resulting in over-segmentation (D1) and under-segmentation (D2). On the other hand, referring to FIG. 8, it is confirmed that the method according to the present disclosure produces the result more similar to the reference standard.

This improvement appears to be due to the consideration of at least one of the consensus map or the spatial feature of the medical image.

As described above, according to the present disclosure, a diffusion model, which is effective in learning complex data distributions, is used to segment medical images, enabling precise segmentation. Furthermore, the segmentation accuracy of bone structures with unclear boundaries may be improved.

The program described above is configured such that the processor (CPU) of the computer is a device of the computer, so that the computer reads the program and executes the methods implemented as the program. The code may include code coded in a computer language such as C, C++, JAVA, or machine language that may be read through the interface. Such code may include functional code related to functions that define functions necessary to execute the above methods, and may include control code related to execution procedures necessary for the processor of the computer to execute the functions according to a predetermined procedure. In addition, such code may further include memory reference-related code regarding which location (address) of the internal or external memory of the computer should reference additional information or media necessary for the processor of the computer to execute the functions. In addition, if the processor of the computer needs to communicate with any other remote computer or server to execute the functions, the code may further include communication-related code regarding how to communicate with any other remote computer or server using the communication module of the computer, and what information or media should be sent and received during communication.

The storage medium refers to a medium that stores data semi-permanently and may be read by a device, rather than a medium that stores data for a short period of time, such as a register, cache, or memory. Specifically, examples of the storage medium include, but are not limited to, ROM, RAM, CD-ROM, magnetic tape, floppy disk, and optical data storage devices. That is, the program may be stored in various recording media on various servers accessible to the computer or in various recording media on the user's computer. In addition, the medium may be distributed across network-connected computer systems, so that computer-readable code may be stored in a distributed manner.

The steps of the method or algorithm described in connection with the embodiments of the present disclosure may be implemented directly in hardware, implemented as a software module executed by hardware, or implemented by a combination thereof. The software module may reside in random access memory (RAM), read only memory (ROM), erasable programmable ROM (EPROM), electrically erasable programmable ROM (EEPROM), a flash memory, a hard disk, a removable disk, CD-ROM, or any other form of computer-readable recording medium well known in the art to which the present disclosure pertains.

Although the embodiments of the present disclosure have been described above with reference to the accompanying drawings, those skilled in the art will understand that the present disclosure may be implemented in other specific forms without changing the technical spirit or essential characteristics thereof. Therefore, it should be understood that the embodiments described above are illustrative in all respects and not restrictive.

According to the present disclosure, a medical image can be segmented using a diffusion model that is effective in learning complex data distributions, thereby enabling precise segmentation, and further improving the segmentation accuracy of bone structures with unclear boundaries.

The effects of the present disclosure are not limited to the effects mentioned above, and other effects not mentioned will be clearly understood by those skilled in the art from the description.

What is claimed is:

1. A segmentation device based on a conditional diffusion model utilizing an annotation mask, comprising:
a communication module configured to communicating with an external device;
a memory configured to store at least one process for performing a segmentation operation based on a conditional diffusion model using an annotation mask; and
a processor configured to perform the segmentation operation based on a conditional diffusion model using an annotation mask according to the process,
wherein the processor is configured to:
collect a medical image acquired by photographing a specific part of a patient, input the medical image into a diffusion model trained based on at least one annotation mask,
generate a consensus map indicating a degree of agreement between multiple segmentation result based on the multiple segmentation result being generated from the diffusion model, and
perform correction based on at least one of the consensus map or the medical image and generate a final segmentation result.

2. The device according to claim 1,
wherein the at least one annotation mask is collected by a single annotator or by multiple annotators.

3. The device according to claim 1,
wherein the consensus map represents the degree of agreement of each pixel by integrating the multiple segmentation result generated by the diffusion model, and is converted into a probability map for use.

4. The device according to claim 1,
wherein the diffusion model is a transformer-based conditional diffusion model that uses an image feature of the medical image as conditional information, and extracts diffusion noise and semantic feature through a conditional U-Net framework.

5. The device according to claim 1,
wherein the processor is configured to generate the final segmentation result by performing correction by minimizing an energy function.

6. The device according to claim 1,
wherein the processor is configured to:
when performing correction based on the consensus map and the feature of the medical image to be segmented,
reflect a segmentation match based on a probability for each pixel that is be extracted from the consensus map.

7. The device according to claim 1,
wherein the processor is configured to:
when performing correction based on at least one of the consensus map or the medical image,
perform correction based on an energy function defined in [Equation] below,
$$E ( x ) = {\sum }_{i} {ψ}_{i} ( {x}_{i} ) + {\sum }_{i , j} {ψ}_{ij} ( {x}_{i} , {x}_{j} ) \qquad [ Equation ]$$
herein, E(x) is an energy function, ψᵢ(xᵢ) is a unary potential for each pixel xᵢ, and ψᵢⱼ(xᵢ,xⱼ) is a pairwise potential.

8. The device according to claim 1,
wherein the processor is configured to:
when performing correction based on the consensus map and the feature of the medical image to be segmented,
apply at least one pairwise pixel feature of the consensus map or the medical image, including at least one of spatial proximity, intensity similarity, or gradient directionality.

9. A segmentation method based on a conditional diffusion model utilizing an annotation mask, the method performed by a device comprising:
collecting a medical image acquired by photographing a specific part of a patient;
inputting the medical image into a diffusion model trained based on at least one annotation mask;
generating a consensus map indicating a degree of agreement between multiple segmentation result based on the multiple segmentation result being generated from the diffusion model, and
performing correction based on at least one of the consensus map or the medical image and generating a final segmentation result.

10. The method according to claim 9,
wherein the at least one annotation mask is collected by a single annotator or by multiple annotators.

11. The method according to claim 9,
wherein the consensus map represents the degree of agreement of each pixel by integrating the multiple segmentation result generated by the diffusion model, and is converted into a probability map for use.

12. The method according to claim 9,
wherein the diffusion model is a transformer-based conditional diffusion model that uses an image feature of the medical image as conditional information, and extracts diffusion noise and semantic feature through a conditional U-Net framework.

13. The method according to claim 9,
wherein generating the final segmentation result includes generating the final segmentation result by performing correction by minimizing an energy function.

14. The method according to claim 9,
wherein generating the final segmentation result includes:
when performing correction based on the consensus map and the feature of the medical image to be segmented,
reflecting a segmentation match based on a probability for each pixel that is be extracted from the consensus map.

15. The method according to claim 1,
wherein generating the final segmentation result includes:
when performing correction based on at least one of the consensus map or the medical image,
performing correction based on an energy function defined in [Equation 1] below,
$$E ( x ) = {\sum }_{i} {ψ}_{i} ( {x}_{i} ) + {\sum }_{i , j} {ψ}_{ij} ( {x}_{i} , {x}_{j} ) \qquad [ Equation 1 ]$$
herein, E(x) is an energy function, ψᵢ(xᵢ) is a unary potential for each pixel xᵢ, and ψᵢⱼ(xᵢ,xⱼ) is a pairwise potential.

16. The method according to claim 9,
wherein generating the final segmentation result includes:
when performing correction based on the consensus map and the feature of the medical image to be segmented,
applying at least one pairwise pixel feature of the consensus map or the medical image, including at least one of spatial proximity, intensity similarity, or gradient directionality.

17. A computer-readable recording medium storing a program for executing the segmentation method based on a conditional diffusion model utilizing an annotation mask according to claim 9.
